(19) United States
(12) Patent Application Publication
(10) Pub. No.: US 2006/0281949 A1
(43) Pub. Date: Dec. 14, 2006

(54) Method for production $g(a),$g(b)-unsaturated amide compounds

(76) Inventors: Beal Weber, Zofingen (CH); Norbert Scharer, Oberentfelden (CH); Beat W. Muller, Therwil (CH)

Correspondence Address: HOFFMANN & BARON, LLP, 6900 JERICHO TURNPIKE, SYOSSET, NY, 11791, US

(21) Appl. No.: 10/565,446

(22) PCT Filed: Jun. 29, 2004
(86) PCT No.: PCT/CH04/00408

§ 371 Date: Jul. 13, 2006

(30) Foreign Application Priority Data
Jul. 21, 2003 (CH) 12874/03

(51) Int. Cl. C07C 231/12

(52) U.S. Cl. 564/123; 564/192; 564/171

(57) ABSTRACT

The invention relates to a method for production of α,β-unsaturated amide compounds of general formula (I): whereby (A) a protective group is introduced into a molecule of general formula (II) to give a compound of formula (III), (B) the compound obtained is reacted in the presence of (i) a dehydrogenation catalyst and (ii) a suitable oxidation agent and (C) the protective groups are removed.

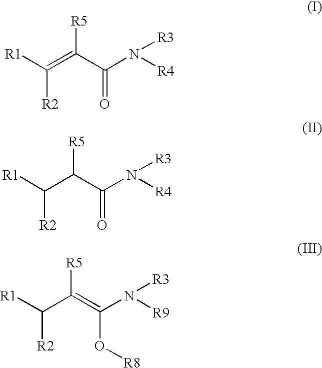

The present invention relates to methods for producing α,β-unsaturated amide compounds or methods for introducing an α,β-unsaturated double bond in compounds, which contain an amide grouping by dehydrating the corresponding saturated amide bond in the α,β-position.

The present invention relates to methods for production of α,β-unsaturated amide compounds having the general formula (I): 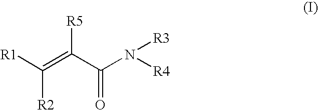
wherein

R₁ and R₂ are independently hydrogen; optionally linear or branched (C₁-C₁₈) alkyl or (C₁-C₁₈) alkenyl substituted with hydroxy, halogen, phenyl, substituted phenyl or an ester group [—C(O)OAlkyl] or an amide group [—C(O)NH₂ or —C(O)NHAlkyl); optionally phenyl substituted with halogen;

or

R₁ or R₂ is a group Y—R₆; wherein

Y is oxygen (—O—); sulphur (—S—); —NR₇—; or dialkylsilyloxy [—(alkyl)₂Si—O—];

R₆ is hydrogen, linear or branched (C₁-C₁₈) alkyl substituted optionally with hydroxy, halogen, phenyl, substituted phenyl or with an ester group [—C(O)OAlkyl] or an amide group [—C(O)NH₂] or

[—C(O)NHAlkyl]; optionally phenyl substituted with halogen;

R₇ is (C₁-C₁₈) alkyl or —N(R₆)(R₇) is a 5- or 6-membered heterocyclic ring;

or

R₁ together with R₃ is directly bonded or forms a group of the formula —(CH₂)ₙ—; wherein

n is a whole number from 1 to 12;

or

R₁ together with R₂ is cyclohexylidene;

or

R₁ together with R₅ and the incorporated (C═C)-double bond is cyclohexenyl;

or

R₁ together with R₅ and the incorporated (C═C)-double bond forms a group of a mono-unsaturated bi-cyclic ring;

R₃ is hydrogen, optionally linear or branched (C₁-C₁₂) alkyl substituted with phenyl, hydroxyl, or halogen, optionally carrying one or more oxygen atoms, (C₅-C₈)-cycloalkyl or (C₅-C₈)-cycloalkenyl, optionally carrying one or more oxygen atoms; optionally phenyl substituted with halogen or hydroxyl; or R₃ together with R₁ is directly bonded or forms a group of the formula —(CH₂)ₙ—;

R₄ has one of the meanings of R₃, preferably hydrogen, optionally linear or branched (C₁-C₁₂) alkyl substituted with phenyl, hydroxyl, or halogen, optionally phenyl substituted with halogen or hydroxyl; or

—NR₃R₄ is a 5- or 6-membered heterocyclic ring; and

R₅ has one of the meanings specified for R₁ or R₂ as independent substituents [i.e. hydrogen; optionally linear or branched (C₁-C₁₈) alkyl or (C₁-C₁₈)-Alkenyl substituted with hydroxy, halogen, phenyl, substituted phenyl, or an ester group [—C(O)OAlkyl] or an amide group [—C(O)NH₂ or —C(O)NHAlkyl]; or optionally, phenyl substituted with halogen];

wherein said method comprises the steps of:

(A) reacting a compound of the general formula (II):

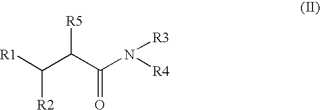

wherein R₁, R₂, R₃, R₄ and R₅ have the meanings given above, to introduce protective groups, so as to produce a compound with the general formula (III): 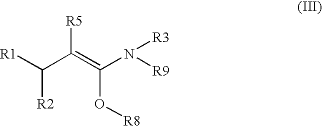
wherein
R₈ is trialkylsilyl, or (when R₄=hydrogen) together with R₉ forms the group —C(O)—(CH₂)ₘ—C(O)— and
R₉ (when R₄=hydrogen) is alkyloxycarbonyl or phenyloxycarbonyl, preferably Boc (=tert. butyloxy-carbonyl); or trialkylsilyl, or together with R₈ the group —C(O)—(CH₂)ₘ—C(O)—, and
m is 0, 1, 2, or 3, preferably 0 or 1, preferably 0, and in the case in which for the compound of the general formula (II), hydroxyl is present, it is optionally reacted with a monovalent protective group R₈ and/or R₉;

(B) reacting the compound obtained in step (A) in presence of (i) a dehydrogenation catalyst and in presence of (ii) a suitable oxidising agent, such as optionally substituted benzoquinone, allylmethyl carbonate, allylethyl carbonate and/or allylpropyl carbonate, to introduce an α,β-double bond in the α,β-position, and

(C) optionally, if present, removing the protective groups R₈, as well as the substituent R₉.

Suitable oxidising agents [in step (B)] include organic as well as inorganic compounds which form palladium compounds of the oxidation state +II from palladium compounds of the oxidation state zero. For example, allyl methyl carbonate reacts, as is known from the literature (Tetrahedron Letters, Vol. 25., No 42, 4783-4786, 1984) through oxidative addition at palladium(0) to form the corresponding palladium(II) allyl derivatives. Other oxidising agents with a similar effect are known to the person skilled in the art. It must be mentioned that in step (B) the substituent R₈ bonded to the amide unit through oxygen is removed at the same time.

R₁ and R₂ are independently, preferably hydrogen; optionally linear or branched (C₁-C₈) alkyl or (C₁-C₈) alkenyl, substituted with hydroxy, phenyl, with halogen or hydroxy substituted phenyl, or with an (C₁₋₄)alkyl ester group [—C(O)O(C₁₋₄)alkyl] or an amide group [—C(O)NH₂] or (C₁₋₄) alkyl amide group [—C(O)NH(C₁₋₄)alkyl]; preferably, phenyl substituted with halogen; preferably linear or branched (C₁-C₈) alkyl or (C₁-C₈) alkenyl; benzyl or phenyl.

Preferably, R₂ is hydrogen and R₁ is preferably linear or branched (C₁-C₈) alkyl or (C₁-C₈) alkenyl; benzyl or phenyl or Y—R₆, where the definitions and constraints given further below are applicable for Y—R₆ or R₁ is hydrogen and R₂ has the broader meaning (specified for R₁).

Preferred are also the meanings, in which R₁ together with R₃ is directly bonded or is a group of the formula —(CH₂)ₙ— and n is a whole number from 1 to 12; or R₁ together with R₂ stands for cyclohexylidene; or R₁ together with R₅ cyclohexenyl.

If either R₁ or R₂ stands for a group Y—R₆, then Y is preferably oxygen (—O—).

If R₁ together with R₃ is directly bonded or forms a group of the formula —(CH₂)ₙ—, then the compound of the formula (I) preferably stands for a lactam of an omega amino fatty acid, for example omega amino butyric acid (ω- butyrolactam), omega amino valeric acid (ω-valero-lactam), omega amino capronic acid (ω-caprolactam), or the omega amino lauric acid (ω-laurinolactam), which have an α,β-unsaturated double bond as per the compound of the general formula (I).

If R₁ together with R₅ and the incorporated (C═C)-double bond represents a monounsaturated bicyclic ring, then it is preferably a norbornyl group optionally substituted with hydroxyl or amino, preferably a norbornyl group.

R₃ preferably stands for hydrogen, optionally linear or branched (C₁-C₄) alkyl, cyclohexyl, substituted with phenyl; phenyl; or R₃ together with R₁ is directly bonded or forms a group of the formula —(CH₂)ₙ—.

R₄ preferably stands for hydrogen, optionally linear or branched (C₁-C₄) alkyl or phenyl substituted with phenyl, preferably hydrogen.

The group —NR₃R₄ is a heterocyclic ring preferably a pyrrolidine or piperidine.

R₅ preferably stands for hydrogen, tertiary butyl or phenyl substituted with halogen or hydroxyl, preferably hydrogen.

R₆ is preferably hydrogen, optionally linear or branched (C₁-C₈) alkyl substituted with hydroxy, halogen, phenyl, with halogen substituted phenyl, or with an (C₁₋₄) alkyl ester group [—C(O)O(C₁₋₄) alkyl] or an amide group [—C(O)NH₂] or (C₁₋₄)alkyl amide group [—C(O)NH(C₁₋₄) alkyl]; optionally phenyl substituted with halogen; preferably hydrogen, optionally linear or branched (C₁-C₈) alkyl substituted with phenyl or with an (C₁₋₄)alkyl ester group or an amide group or an (C₁₋₄)alkyl amide group; or phenyl; preferably hydrogen, linear or branched (C₁-C₈)alkyl or phenyl.

R₇ preferably stands for (C₁-C₈) alkyl. The substituent N (R₆)(R₇) stands for a heterocyclic ring preferably a pyrrolidine or piperidine group.

R₈ preferably stands for trimethylsilyl, or together with R₉ the group —C(O)—(CH₂)ₘ—C(O)—, in which m stands for 0, 1, 2, or 3, preferably 0 or 1, preferably zero.

R₉ is alkyloxycarbonyl preferably isobutyloxy-carbonyl, tert. butyloxycarbonyl, tert. amyloxycarbonyl, cyclobutyloxycarbonyl, 1-methylcylobutyloxycarbonyl, cyclopentyloxycarbonyl, cyclohexyloxycarbonyl, 1-methyl-cyclohexyl, preferably tert. butyloxycarbonyl.

Dialkylsilyl preferably stands for dimethylsilyl. Trialkylsilyl preferably stands for trimethylsilyl. Halogen preferably stands for fluorine or chlorine, preferably fluorine. An alkyl ester group preferably stands for a methyl-, ethyl-, propyl- or butylester group. An alkyl amide group preferably stands for a methyl-, ethyl-, propyl- or butyl amide group.

Compounds, which are produced as per the invention and which can be included under the general formula (I) are, for instance, the corresponding α,β-unsaturated compounds of N,N-dialkyl alkylamides, such as N,N-dimethylbutylamide and homologous compounds, or of the lactams mentioned earlier. Other examples for production of α,β-unsaturated amide compounds as per the invention are:

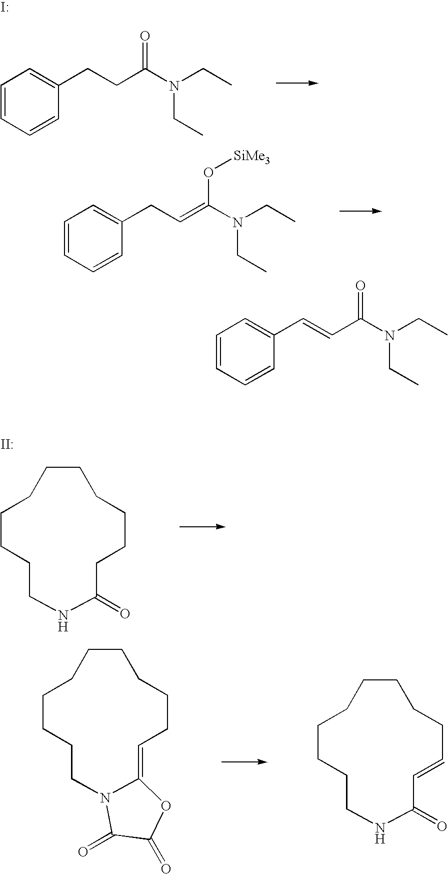

For introducing the protective group trialkylsilyl, i.e. for silylation of the NH-group and/or of the oxygen atom or the OH group [as per step (A)], one preferably uses an (alkyl)₃Si(halogen), such as (CH₃)₃SiCl, or bis-trimethyl-silyltrihalogen acetamide, bistrimethylsilyl acetamide, hexamethyldisilazane and/or bistrimethyl urea, preferably bistrimethylsilyl-trifluoroacetamide, or a trialkylsilyl-trifluoromethane sulphonate, preferably trimethylsilyl-trifluoromethane sulphonate. The reaction conditions for the silylation are known from EP 0 473 226.

For introducing a protective group, in which R₇ together with R₈ stands for the group —C(O)—(CH₂)ₘ13 C(O)— and in which m has the notations specified earlier, one converts the compound of the general formula (II) or the lactam grouping [as per step (A)] with the corresponding dihalogenide, preferably oxalylchloride (oxalic acid chloride) or malonyl chloride (malonic acid chloride), where oxalyl chloride is preferred. The reaction conditions for the conversion with oxalyl chloride are known from EP 0 428 366 and are to be used for the conversion with malonyl chloride or in the same way for similar reacting compounds.

For introducing a protective group, in which R₈ stands for alkyloxy carbonyl, such as tert. butyloxycarbonyl (Boc), one proceeds in a known way by converting the compounds of the general formula (II) with for example Boc anhydride (Boc O-Boc) {[(CH₃)₃C—O—C(O)]₂—O} or with Boc carbamate [(CH₃)₃C—O—C(O)—N(C₁₋₄ alkyl )₂]. Thereby, Boc is the representative for compounds reacting in a similar way, that is the compounds, in which the tert. butyl group is replaced by another similar reacting group, such as the mentioned groups of tert. amyl, cyclobutyl, cyclopentyl or cyclohexyl. Such analogous reactions find numerous mentions in the technical literature. If R₈ stands for trialklylsilyl and R₉ for Boc, then one introduces first the protective group Boc and thereafter silylates.

In step (B) the compound obtained in step (A) is reacted in the presence of (i) a dehydrogenation catalyst and (ii) in the presence of a suitable oxidising agent, like optionally substituted benzoquinone, allyl methyl carbonate, allyl ethyl carbonate and/or allyl propyl carbonate, to introduce the α,β-double bond in the α,β position.

The dehydrogenation catalyst is selected preferably from compounds (salts and complexes) of the group of the transition metals of the periodic system, in particular from the compounds of the metals of the group VIII. of the periodic system, in particular from iron (Fe), ruthenium (Ru) and osmium (Os); cobalt (Co), rhodium (Rh), and iridium (Ir); nickel (Ni), palladium (Pd) and platinum (Pt) as well as the group IB, i.e. of copper (Cu), silver (Ag) and gold (Au). Preferred are the compounds of the metals of the group VIII of the periodic system. Preferred are especially compounds or dehydrogenation catalysts based on rhodium (Rh), palladium (Pd) and platinum (Pt). Preferred are palladium compounds. Examples of such palladium compounds are: Pd(0)-compounds such as tris(dibenzylidene acetone)-dipalladium chloroform complex and Pd(II) compounds such as PdCl₂, Pd(dppe)₂, [dppe=bis-(1, 2-biphenylphosphino)ethane], Pd(dppe)Cl₂, Pd(OAc)₂, Pd(dppe) (OAc)₂, π-allyl Pd-complexes, preferably π-allyl Pd chloride dimer. Preferred are Pd(0) compounds, especially tris (dibenzylidene acetone) dipalladium chloroform complex. These compounds, or salts and complexes, are well known and are described in the literature.

For thermal stabilisation of the palladium complex an additional complexing agent such as 2,2′-bipyridyl or 1,10-phenanthroline can be used, preferably 2,2′-bipyridyl.

As quinone, one can also use a substituted quinone, such as a quinone substituted with C₁₋₄ alkyl, halogen, cyano or nitro. Such quinones are well known.

For explanation, it can be added for the mechanism of the catalysis, that a Pd-species adds at the C-atom in 2-position under splitting of the oxygen protective group [e.g. the —Si(CH₃)₃-group]. A subsequent beta-hydrogen splitting at the C-atom in 1-position leads to the desired Δ¹-double bond in 1-/2-position, and releases another palladium species, which is fed back in the catalytic cycle. Instructions for this reaction mechanism are given in the Tetrahydron Letters, page 4783, (1984). However, the present invention does not relate to this explanation.

In step (C) the compound obtained is then converted to the compound having the formula (I) by removal of the protective groups. This takes place preferably through a treatment with a suitable acid, such as with formic acid, acetic acid and/or trifluoroacetic acid, preferably with formic acid. Methods for isolating the compounds of the general formula (I) from the reaction mixture as well as for their further purification are known to persons skilled in the art. Thereafter, the compounds obtained can be further processed.

For the described methods with the steps (A)-(C), numerous dry organic solvents, such as toluene, benzine, hexane, heptane, tert. butyl alcohol, diethylether, acetone, benzene, dioxane, tetrahydrofuran, chloroform, dimethylformamide or pyridine, may be used which are free of water.

The following examples illustrate the invention.

### EXAMPLE 1

### Production of an α,β-unsaturated butyramide, i.e. but-2-enoic acid dimethylamide

### Step 1A (Production of butyramide silylenolether, i.e. dimethyl-(1-trimethylsilanyloxy-but-1-enyl)-amine)

46 ml of a 2 molar (2M) lithium di-isopropylamide solution (LDA solution) is added carefully to a solution of 10 g (0.085 Mol) N,N-dimethylbutyramide and 54 g absolute tetrahydrofuran (THF) at an internal temperature of −80° C. and stirred for about 1 hour at −70 to −80° C. Thereafter, at the same internal temperature 28 g (0.255 Mol) tri-methylchlorosilane is added. Thereby, LiCl precipitates out. After adding the silane the cold bath is removed. One lets the mixture warm up to the ambient temperature overnight under nitrogen (N₂). At an internal temperature of 70-90° C. the reaction mixture is distilled under N₂ flow, thereby about 8 g of the desired silyl enol ether accumulates in the sample.

¹H-NMR (200 MHz, CDCl₃, δ): 3.48-3.38 (1H, t); 2.28(6H, s); 1.89-1.72(2H, m); 0.77 (3H, t); 0.02 (9H, s)

### Step 1B (Production of α,β-unsaturated butyramide, i.e. but-2-enoic acid dimethylamide)

2 g (8 mMol) of the silyl enol ether from step 1 is heated under nitrogen with 16 g absolute acetonitrile, 2 g chloroform, 2.9 g (0.024 Mol) allyl methyl carbonate and 0.16 g (0.16 mMol) of the Pd catalyst to the reflux temperature (internal temperature 75-80° C.). Already during heating, a clearly visible gas formation sets in. The dark green solution is stirred overnight. The black suspension thus obtained is filtered and is concentrated under reduced pressure (only up to p=80 mbar). One gets about 0.9 g of unsaturated butyramide.

¹H-NMR (200 MHz, CDCl₃, δ): 6.88-6.72 (1H, m); 6.20(1H, d broad); 3.04 (3H, s); 2.98 (3H, s); 1.78(3H, d); MS (+EI): 114 (M+1, 40%); 98 (100%)

In the same way, as described above, 4-dimethylcarbamoyl-2,2-dimethyl-butyric acid methyl ester can be converted into 4-dimethylcarbamoyl-2,2-dimethyl-but-3-enoic acid methyl ester.

### EXAMPLE 2

### Production of α,β-unsaturated valerolactam, i.e. 6-oxo-3,6-dihydro-2H-pyridine-1-carboxylic acid tert-butyl ester

### Step 2A (Production of N-bocylated valerolactam, i.e. 2-oxo-piperidine-1-carboxylic acid tert-butyl ester)

55 ml of a 2M LDA solution is added carefully to a solution of 10 g (0.097 Mol) δ-valerolactam and 44.5 g absolute THF at an internal temperature of −60° C. and stirred for about 1 hour at −60 to −70° C. Thereafter, at the same internal temperature, one adds drop wise a solution comprising 22.22 g (0.102 mol) boc anhydride and 18 g absolute THF and lets the reaction mixture warm up to the ambient temperature overnight. The mixture thus obtained is added to a mixture comprising 50 g toluene and 100 g water and stirred for about 30 minutes. The red, organic phase is washed three times each with 50 g water and then concentrated through distillation, as far as possible, under reduced pressure. This results in 19 g of a dark oil.

¹H-NMR (200 MHz, CDCl₃, δ): 3.78-3.55 (2H, t); 2.55-2.42(2H, t); 1.90-1.72 (4H, m); 1.57-1.48 (9H, s)

MS: 199 (M, <1%); 144 (46%); 57 (100%)

### Step 2B (Production of boc-valerolactam-silyl enol ether, i.e. 6-trimethylsilanyloxy-3,4-dihydro-2H-pyridine-1-carboxylic acid tert-butyl ester)

30 ml of a 2M LDA solution is added carefully to a solution comprising 12 g (0.052 mol) of N-boc-valero-lactam from step 1 and 44.5 g of absolute THF at an internal temperature of −60° C. and stirred for about 1 hour at −60° C. to −70° C. Thereafter, at the same internal temperature, 6.2 g (0.057 mol) of trimethylchlorosilane is added. LiCl thereby precipitates out. After adding the silane, the cold bath is removed. One lets the mixture warm up to the ambient temperature under N₂. The reaction mixture is then poured into a mixture comprising 50 g toluene and 50 g water, stirred briefly and the organic phase is washed three times, each time with 50 g water. After concentration, 14 g of a clear oil remains in the flask.

¹H-NMR (200 MHz, CDCl₃, δ): 4.12 (1H, t); 3.03 (2H, t); 1.92-1.81 (2H, m); 1.55-1.40 (2H, m); 1.27(9H, s); 0.01 (9H, s)

### Step 2C (α,β-unsaturated valerolactam, i.e. 6-oxo-3,6-dihydro-2H-pyridine-1-carboxylic acid tert-butyl ester)

2g (0.0074 Mol) of the silyl enol ether from state 2 is stirred together with 25 g of absolute acetonitrile, 0.4 g Pd catalyst and 0.8 g p-benzoquinone overnight at room temperature. The black reaction mixture obtained is vigorously stirred with 50 g of 5% NaOH solution, extracted with 50g of toluene and the organic phase is concentrated as far as possible. About 1 g of freely moving, dark oil remains in the flask.

¹H-NMR (200 MHz, CDCl₃, δ): 6.82-6.72 (1H, m); 5.97 (1H, d); 3.88 (2H, t); 2.46-2.35 (2H, m); 1.54 (9H, s)

### EXAMPLE 3

### N,N-diethyl-3-phenyl-acrylamide

### Step 3A (N,N-diethyl-(3-phenyl-l-trimethylsilanyloxy-propenyl)amine)

In 15 ml of tetrahydrofuran 3.3 ml of di-isopropylamine is added under cooling (−78° C.) with 9.25 ml of 2.5 M hexyl-lithium solution. After 30 min 4.11 g (20 mmol) of N,N-diethyl-3-phenyl-propionamide is added. After further 60 minutes 7.6 ml of trimethylchlorosilane is added. The reaction mixture is left overnight to warm up to room temperature. Under reduced pressure (approx. 0.6 mbar), 3.55 g (64% of the theor.) N,N- diethyl-(3-phenyl-1-trimethylsilanyloxy-propenyl)amine (intermediate product) is obtained through distillation at 125-128° C., which can be used in step 2 without further purification.

### Step 3B (N,N-diethyl-3-phenyl-acrylamide)

A solution of 20 ml acetonitrile, 1.35 ml chloroform, 2.63 ml allyl methyl carbonate, 0.15 g tris-(dibenzylide-acetone)dipalladium chloroform complex and 2.1 g of the intermediate product made above is boiled overnight at reflux. The reaction mixture is filtered clear and concentrated in vacuum. The remaining group contains 1.2 g N,N-diethyl-3-phenyl-acrylamide.

MS (eI): 204 (5%), 203 (M+, 35%), 188 (18%), 131 (100%)

### EXAMPLE 4

### azacyclotridec-3-en-2-on

### Step 4A (4,5,6,7,8,9,10,11,12,13-decahydro-1-oxa-3a-aza-cyclopentacyclotridecene-2,3-dione)

At room temperature, 19 g (0.13 mol) of oxalyl chloride is added to a solution of 19.7 g (0.1 mol) of laurino-lactam in 400 ml toluene. The reaction mixture is stirred for 3 hours at 55° C. and subsequently concentrated in vacuum. The group is crystallised with 300 ml heptane. About 21.4 g (85% of the theor.) of the intermediate product (4,5,6,7,8,9,10,11,12,13-decahydro-1-oxa-3a-aza-cyclopentacyclotridecene-2,3-dione) is obtained.

¹H-NMR (200 MHz, CDCl₃, δ): 5.11 (2H, t); 3.73-3.67 (2H, m) 2.25-2.20 (2H, m); 1.70-1.29 (16H, m).

¹³C-NMR (50 MHz, CDCl₃, δ): 156.4; 151.6; 142.0; 40.4; 28.4; 27.4; 26.8; 26.7; 25.9; 25.4; 24.4; 24.1; 23.9.

### Step 4B (azacyclotridec-3-en-2-on)

A mixture of 1.0 g(4 mmol) of the just produced intermediate product (4,5,6,7,8,9,10,11,12,13-decahydro-1-oxa-3a-aza-cyclopentacyclotridecene-2,3-dione), 1.4 g of allyl methyl carbonate, 5.9 g chloroform, 0.1 g tris-(dibenzylide acetone)dipalladium chloroform complex and 10 ml acetonitrile are boiled at reflux. After 4 hours and 16 hours additional 0.1 g tris-(dibenzylidenacetone)-dipalladium chloroform complex is added. The reaction mixture is concentrated after 2 days of boiling, dissolved in 20 ml of methanol and stirred at 0° C. with 8 mmol sodium methylate (dissolved in 1.5 ml methanol) during 1 hour, and then concentrated in vacuum. The residue is diluted with acetic acid ethyl ester and washed with 1 N hydrochloric acid. The organic phase is concentrated.

About 1.2 g of group remains comprising 6% laurinolactam, 5% azacyclotridec-3-en-2-on, 11% 3-allyl azacyclotridecan-2-on, 47% intermediate product (4,5,6,7,8,9,10,11,12,13-decahydro-1-oxa-3a-aza-cyclo-pentacyclotridecene-2,3-dione), 25% 14-allyl 4,5,6,7,8,9,10,11,12,13-decahydro-1-oxa-3a-aza-cyclopentacyclo-tri-decene-2,3-dione and 6% dibenzylidene acetone. The products are separated chromatographically (silica gel, acetic acid ethyl ester/hexane).

Azacyclotridec-3-en-2-on: MS (eI): 195 (M⁺, 18%), 167 (18%), 152 (18%), 150 (20%), 124 (46%), 81 (100%). 3-allyl azacyclotridecan-2-on: MS (eI): 237 (M⁺, 50%), 207 (38%), 196 (65%), 55 (100%).

14-allyl 4,5,6,7,8,9,10,11,12,13-decahydro-1-oxa-3a-aza-cyclopentacyclotridecene-2,3-dione: ¹H-NMR (200 MHz, CDCl₃, δ): 5.5.3 (1H, ddt), 5.09 (1H, d); 5.08 (1H, d); 3.85 (1H, ddd); 3.76 (1H, ddd); 2.57 (1H, dd); 2.49 (1H, dd); 2.56-1.00 (18H, m).

MS (eI): 250 (M⁺-allyl, 80%), 207 (100%).

In a similar way, the α,β-unsaturated compound 4a,5,6,7,8,8a-hexahydro-1H-quinolin-2-on can be obtained from octahydro-quinoline-2-on.

1. Method for the production of α,β-unsaturated amide compounds having the general formula (I): 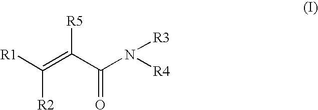
wherein,
R₁ and R₂ are independently hydrogen; optionally linear or branched (C₁-C₁₈) alkyl or (C₁-C₁₈) alkenyl substituted with hydroxy, halogen, phenyl, substituted phenyl, or an ester group [-C(O)Oalkyl] or an amide group [—C(O)NH₂ or —C(O)NHalkyl]; optionally phenyl substituted with halogen;
or
R₁ or R₂ comprises a group Y—R₆; in which
Y is oxygen (—O—); sulphur (—S—); —NR₇—; or dialkylsilyloxy [—(alkyl)₂Si—O—];
R₆ is hydrogen, optionally linear or branched (C₁-C₁₈) alkyl substituted with hydroxy, halogen, phenyl, substituted phenyl or with an ester group [—C(O)OAlkyl] or an amide group [—C(O)NH₂] or [—C(O)NHAlkyl]; optionally phenyl substituted with halogen;
R₇ is (C₁-C₁₈) alkyl or —N(R₆)(R₇) is a 5- or 6-membered heterocyclic ring;
or
R₁ together with R₃ is directly bonded or a group having the formula —(CH₂)ₙ—; in which n is a whole number from 1 to 12;
or
R₁ together with R₂ is cyclohexylidene;
or
R₁ together with R₅ and the incorporated (C═C)-double bond is cyclohexenyl;
or
R₁ together with R₅ and the incorporated (C═C)-double bond forms a group of a monounsaturated bicyclic ring;
R₃ is hydrogen, optionally a linear or branched (C₁-C₁₂) alkyl substituted with phenyl, hydroxyl, or halogen, carrying one or more oxygen atoms, (C₅-C8)-cycloalkyl or (C₅-C8)-cycloalkenyl, carrying one or more oxygen atoms; preferably, phenyl substituted with halogen or hydroxyl; or R₃ together with R₁ is directly bond or forms a group of the formula —(CH₂)ₙ—;
R₄ has one of the meanings of R₃, preferably hydrogen, optionally linear or branched (C₁-C ₁₂) alkyl substituted with phenyl, hydroxyl, or halogen, optionally phenyl substituted with halogen or hydroxyl; or
—NR₃R₄ a 5- or 6-membered heterocyclic ring; and
R₅ has one of the meanings specified for R₁ or R₂ as independent substituents, wherein said method comprises the steps of:
(A) reacting a compound of the general formula (II): 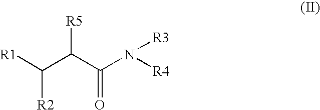
wherein R₁, R₂, R₃, R₄ and R₅ have the meanings specified above, to introduce protective groups so as to produce a compound of the general formula (III):
wherein R₈ is trialkylsilyl, or (when R₄=hydrogen) together with R₉ forms the group —C(O)—(CH₂)ₘ—C(O)— and
R₉ (when R₄=hydrogen) is alkyloxycarbonyl or phenyloxycarbonyl, preferably Boc (=tert. butyloxy-carbonyl); or trialkylsilyl, or together with R₈ the group —C(O)—(CH₂)ₘ—C(O)—, and
m is 0, 1, 2, or 3, preferably 0 or 1, preferably 0, and in the case in which for the compound of the general formula (II) hydroxyl is present, it is reacted, with a monovalent protective group R₈ and/or R₉;
(B) reacting the compound obtained in step (A) in the presence of (i) a dehydrogenation catalyst and in the presence of (ii) an oxidising agent, such as optionally substituted benzoquinone, allyl methyl carbonate, allyl ethyl carbonate and/or allyl propyl carbonate, to introduce an α,β-double bond in the α,β-position, and
(C) optionally removing, if present, the protective groups R₈, as well as the substituent R₉.

2. Method according to claim 1, wherein R₁ and R₂ are independently hydrogen, optionally linear or branched (C₁-C₈) alkyl or (C₁-C₈) alkenyl substituted with hydroxy, phenyl, phenyl substituted with halogen or hydroxy, or with a (C₁₋₄) alkyl ester group or an amide group or (C₁₋₄) alkyl amide group, preferably, phenyl substituted with halogen; preferably linear or branched (C₁-C₈) alkyl or (C₁-C₈) alkenyl, benzyl or phenyl.

3. Method according to claim 1, wherein R₂ is hydrogen and R₁ is linear or branched (C₁-C₈) alkyl or (C₁-C₈) alkenyl, benzyl or phenyl or Y—R₆.

4. Method according to claim 1, wherein R₁ is hydrogen and R₂ is linear or branched (C₁-C₈) alkyl or (C₁-C₈) alkenyl; benzyl or phenyl or Y—R₆.

5. Method according to claim 1, wherein R₁ together with R₃ is directly bonded or forms a group of the formula —(CH₂)ₙ— and n is a whole number from 1 to 12; or R₁ together with R₂ is cyclohexylidene; or R₁ together with R₅ is cyclohexenyl.

6. Method according to claim 1, wherein Y in the group Y—R₆ is oxygen.

7. Method according to claim 1, wherein R₆ is hydrogen, optionally linear or branched (C₁-C₈) alkyl or phenyl substituted with hydroxy, halogen, phenyl, phenyl substituted with halogen, or an (C₁₋₄)alkyl ester group or an amide group or a (C₁₋₄)alkyl amide group; optionally phenyl substituted with halogen; preferably hydrogen, optionally linear or branched (C₁-C₈) alkyl substituted with phenyl, or with a (C₁₋₄) alkyl ester group or an amide group or a (C₁₋₄) alkyl amide group; or phenyl; preferably hydrogen, linear or branched (C₁-C₈) alkyl or phenyl.

8. Method according to claim 1, wherein the substituent —N(R₆)(R₇) as heterocyclic ring is a pyrrolidine or piperidine.

9. Method according to claim 1, wherein the compound of the formula (II) represents a lactam of an omega amino fatty acid, preferably aminobutyric acid, omega aminovaleric acid, omega aminocapronic acid, or omega aminolauric acid.

10. Method according to claim 1, wherein the compound of the formula (I), R₁ together with R₅ and the incorporated (C═C)-double bond represent a monounsaturated bicyclic ring, preferably a norbornyl group optionally substituted with hydroxyl or amino, preferably a norbornyl group.

11. Method according to claim 1, wherein R₃ and R₄ are independently hydrogen, linear or branched (C₁-C₄) alkyl optionally substituted with phenyl, phenyl; or the group —NR₃R₄ is pyrrolidine or piperidine.

12. Method according to claim 1, wherein R₅ is hydrogen, tert. butyl or optionally phenyl substituted with halogen or hydroxyl, preferably hydrogen; and R₈ is trimethylsilyl or R₈ together with R₉ is the group —C(O)—(CH₂)ₘ—C(O)—; or R₉ is Boc, trimethylsilyl, or R₉ together with R₈ is the group —C(O)—(CH₂)ₘ—C(O)—, in which m is 0, 1, 2, or 3, preferably 0 or 1, preferably 0.

13. Method according to claim 1, wherein R₉ is alkyloxycarbonyl, isobutyloxycarbonyl, tert. butyloxycarbonyl, tertiary amyloxycarbonyl, cyclobutyloxycarbonyl, 1-methylcylobutyloxycarbonyl, cyclopentyloxycarbonyl, cyclohexyloxycarbonyl, 1-methyl-cyclohexyl, preferably tertiary butyloxycarbonyl.

14. Method according to claim 1, wherein the dehydrogenation catalyst [in step (B)] is selected from amongst compounds (salts and complexes) of the transition metals of the periodic system, preferably from compounds of the metals of Group VIII elements, in particular from iron, ruthenium and osmium; cobalt, rhodium, and iridium; nickel, palladium and platinum; copper, silver and gold preferably from compounds based on rhodium, palladium and platinum.

15. Method according to claim 14, wherein the dehydrogenation catalyst is a palladium compound, preferably a Pd(0) compound, preferably a tris(dibenzylidene acetone) dipalladium chloroform complex or a Pd(II) compound, preferably PdCl₂, Pd(dppe)₂, Pd(dppe)Cl₂, Pd(OAc)₂, Pd(dppe)(OAc)₂, π-allyl Pd complex, preferably π-allyl Pd chloride dimer.

16. Method according to claim 1, wherein an additional complexing agent is used for the thermal stabilisation of the palladium complex, preferably 2,2′-bipyridyl or 1,10-phenanthroline.

17. Method according to claim 1, wherein the quinone is a substituted quinone, preferably a quinone substituted with C₁₋₄ alkyl, halogen, cyano or nitro.

18. A compound produced according to the method of claim 1.
